(19) United States
(12) Patent Application Publication
(10) Pub. No.: US 2011/0003393 A1
(43) Pub. Date: Jan. 6, 2011

(54) SYSTEMS AND METHODS FOR STUDYING INFLUENZA

(75) Inventors: James Chou, Cambridge, MA (US); Jason R. Schnell, Cambridge, MA (US)

Correspondence Address: Harvard University & Medical School;c/o Wolf, Greenfield & Sacks, P.C., 600 Atlantic Avenue, Boston, MA, 02210-2206, US

(73) Assignee: President and Fellows of Harvard College, Cambridge, MA (US)

(21) Appl. No.: 12/739,414

(22) PCT Filed: Oct. 24, 2008
(86) PCT No.: PCT/US08/12084

§ 371 Date: Sep. 22, 2010

Related U.S. Application Data
(60) Provisional application No. 61/002,338, filed on Nov. 8, 2007.
(60) Provisional application No. 61/000,386, filed on Oct. 25, 2007.

(51) Int. Cl. G01N 33/50

(52) U.S. Cl. 436/90

(57) ABSTRACT

The present invention generally relates to influenza and, in particular, to systems and methods for studying influenza viruses such as the influenza A virus. One aspect of the invention is generally directed towards systems and methods for determining the timescale dynamics of the tryptophan residue located in position 41 of the M2 proton channel of the influenza A virus, for instance, via nuclear magnetic resonance. This may be useful, for example, in determining whether a candidate drug is able to alter the dynamics of the tryptophan residue, and thus, whether the drug targets the M2 proton channel.

### RELATED APPLICATIONS

This application claims the benefit of U.S. Provisional Patent Application Ser. No. 61/000,386, filed Oct. 25, 2007, entitled “Anti-Influenza Compositions,” by Chou, et al., and U.S. Provisional Patent Application Ser. No. 61/002,338, filed Nov. 8, 2007, entitled “Anti-Influenza Compositions,” by Chou, et al., each incorporated herein by reference.

### GOVERNMENT FUNDING

Research leading to various aspects of the present invention were sponsored, at least in part, by the National Institutes of Health. The U.S. Government has certain rights in the invention.

### FIELD OF INVENTION

The present invention generally relates to influenza and, in particular, to systems and methods for studying influenza viruses such as the influenza A virus.

### BACKGROUND

The integral membrane protein, M2, of the influenza virus forms proton channels in the viral lipid envelope. The low pH of an endosome is believed to activate the M2 channel prior to hemagglutinin-mediated fusion. Conductance of protons acidifies the viral interior and thereby facilitates dissociation of the matrix protein from the viral nucleoproteins, which allows for the unpacking of the viral genome. In addition to its role in the release of viral nucleoproteins, M2 in the membrane of the trans-Golgi network (TGN) can prevent premature conformational rearrangement of newly synthesized hemagglutinin during transport to the cell surface by equilibrating the pH of the TGN with that of the cytoplasm of the infected host cell. Blocking the proton conductance of M2 with the anti-viral drug amantadine or rimantadine thus can inhibit viral replication.

M2 is a 97-residue single-pass membrane protein with its N- and C-termini directed toward the outside and inside of the virion, respectively; it is believed to be a homotetramer in its native state. The channel region is formed by four transmembrane (TM) helices in which His37 is believed to act as a pH sensor and Trp41 is believed to serve as the gate of the channel. Adamantane-based drugs such as amantadine and rimantadine target the M2 channel, and have been used as first-choice antiviral drugs against community outbreaks of influenza A viruses for many years, but resistance to the adamantanes has recently become widespread. However, the mechanism of action for amantadine and rimantadine remains unknown.

### SUMMARY OF THE INVENTION

The present invention generally relates to influenza and, in particular, to systems and methods for studying influenza viruses such as the influenza A virus. The subject matter of the present invention involves, in some cases, interrelated products, alternative solutions to a particular problem, and/or a plurality of different uses of one or more systems and/or articles.

In one aspect, the invention is directed to a method. In one set of embodiments, the method includes an act of determining the dynamics of the tryptophan residue located in position 41 of the M2 proton channel of the influenza A virus via nuclear magnetic resonance. The method, according to another set of embodiments, is generally directed to a method of evaluating a candidate binding species. In one embodiment, the method includes acts of exposing the M2 proton channel of the influenza A virus to a candidate binding species, and determining whether the candidate binding species alters the dynamics of the tryptophan residue located in position 41 of the M2 proton channel of the influenza A virus.

In another aspect, the present invention is directed to a method of making one or more of the embodiments described herein, for example, a method of studying influenza viruses. In another aspect, the present invention is directed to a method of using one or more of the embodiments described herein, for example, a method of studying influenza viruses.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1a is a characterization of the M2(18-60) polypeptide construct, in accordance with one embodiment of the invention.

FIGS. 1b and 1c are gel electrophoresis plots in one embodiment of the invention.

FIG. 1d is an image of the ¹H—¹⁵N transverse relaxation-optimized spectroscopy of tetramer and rimantadine, in yet another embodiment of the invention.

FIGS. 2a and 2b are images of the structure of the M2 channel in the presence of rimantadine, produced in accordance with an embodiment of the invention.

FIG. 2c shows a close-up view of the channel shown in FIGS. 2a and 2b.

FIG. 3a shows the water accesibility of the M2 channel, in yet another embodiment of the invention.

FIG. 3b shows the distribution of the water molecules found within the channel, in still another embodiment of the invention.

FIG. 4a shows the interaction between rimantadine and the M2, according to an embodiment of the invention.

FIG. 4b shows the NOEs between the M2 protein and rimantadine, in another embodiment of the invention.

FIG. 4c is a surface representation of the rimantadine binding pocket, in accordance with yet another embodiment of the invention.

FIG. 5a is an image of the relevant NOEs which broaden upon the lowering of the pH, in accordance with one embodiment of the invention.

FIGS. 5b and 5c are graphs of the R₂ relaxation rate as a function of the frequency of refocusing for the chemical shift evolution, in another embodiment of the invention.

FIGS. 6a and 6b are schematic representations of the M2 channel activation, in still another embodiment of the invention.

FIGS. 7a, 7b and 7c are NMR spectra of reconstituted M2 tetramer in the absence and presence of rimantadine at different pH levels, in an embodiment of the invention.

FIGS. 8, 9, and 10 show detailed NMR spectra of relevant residues of the M2 protein in the presence of rimantadine, in certain embodiments of the invention.

FIG. 11 shows Table 1.

FIG. 12 shows Table 2.

### BRIEF DESCRIPTION OF THE SEQUENCES

SEQ ID NO: 1 is M2, having the sequence

MSLLTEVETPIRNEWGCRCNDSSDPLVVAASIIGILHLILWILDRLFFK
CIYRFFEHGLKRGPSTEGVPESMREEYRKEQQSAVDADDSHFVSIELE.

### DETAILED DESCRIPTION

The present invention generally relates to influenza and, in particular, to systems and methods for studying influenza viruses such as the influenza A virus. One aspect of the invention is generally directed towards systems and methods for determining the timescale dynamics of the tryptophan residue located in position 41 of the M2 proton channel of the influenza A virus, for instance, via nuclear magnetic resonance. This may be useful, for example, in determining whether a candidate drug is able to alter the dynamics of the tryptophan residue, and thus, whether the drug targets the M2 proton channel.

One aspect of the invention is generally directed to systems and methods for studying the M2 proton channel of the influenza A virus, including the tryptophan residue located in position 41. “M2,” as is known to those of ordinary skill in the art, is the integral membrane protein of the influenza A virus. As mentioned, M2 forms proton channels, allowing the passage of protons (H⁺) and/or other ions in the viral lipid envelope, and is believed to facilitate hemagglutinin-mediated fusion of the influenza A virus to occur with the target cell. It is typically activated by low pH, e.g., as may be found in an endosome. M2 itself is a homotetramer, and the units are helixes stabilized by inter-subunit disulfide bonds and/or helix-helix packing forces. The “M2 channel” of M2 refers to the region between the four transmembrane (TM) helices of a tetramer of M2. This channel is used by the influenza virus to shuttle protons between the interior and exterior of the virus. Blocking of proton conductance of this channel is believed to inhibit viral replication. In addition, “tryptophan 41” (Trp41) is the tryptophan residue located at position 41 of M2, and “histidine 37” (His37) is the histidine residue located at position 37. Without wishing to be bound by any theory, it is believed that Trp41 acts as a “gate” that controls proton flow through the M2 channel, and His37 acts as a pH sensor that affects control of the M2 channel in response to the pH of the environment surrounding the M2 channel (e.g., such that low pH, such as within an endosome, is able to activate the M2 channel, as described above). However, it should be understood that the invention is not necessarily limited to the M2 channel. Indeed, the systems and methods as disclosed herein may be applicable in some cases to other membrane proteins, such as other proton channel proteins (e.g., drug-resistant variants of M2) associated with influenza viruses such as the influenza A virus.

As mentioned, and without wishing to be bound by any theory, Trp41 is believed to act as a gate that controls proton flow within the M2 channel. The position of the Trp41 residue may determine whether protons can be conducted through the M2 channel, and/or to what degree protons can pass through the channel. Blocking the proton conductance of the channel may thereby partially, if not completely, inhibit viral replication. Determination of the dynamics of the Trp41 residue may thus help to determine the efficacy of an outside influence, such as a binding species, to limit the proton conductance, and therefore, to at least partially inhibit of viral replication. Thus, one embodiment of the invention is directed to determining the dynamics of the tryptophan residue located in position 41 of the M2 proton channel. The phrase “dynamics of the tryptophan residue at position 41” refers to the change in the degree that the M2 channel is open and allows proton conductance to occur through the channel, as controlled by the Trp41 residue. For example, the channel may be in an open state, a closed state, or any state between an open and closed state. A fully open channel is one in which protons are able to be conducted through the channel at the maximum rate, while a fully closed channel is one in which no protons can be conducted through. In some cases, the channel may change from a relatively open to a relatively closed state, or from a relatively closed to a relatively open state. The degree that the channel is opened or closed can be determined, for instance, using techniques such as those described below. The M2 channel may also be in equilibrium between two different states, in some cases.

The dynamics of the Trp41 residue may be important to determine in some instances, since a decrease in the degree that the channel is open may alter proton conductance within the channel, which could lead to partial or complete inhibition of viral replication. The dynamics of the Trp41 residue may be determined using various techniques, such as the NMR techniques as discussed below.

In some cases, for example in a population of M2 channels, the channels may be open (or closed) to varying degrees, and accordingly, a given M2 channel may have a certain probability of being in an open or a closed state. Accordingly, the phrase “probability of channel opening” refers to the chance or probability of finding the channel in one state (e.g., in an open state) versus finding the channel in another state (e.g., in a closed state). The openness of the M2 channel may be changed by various methods, including, but not limited to, a change in pH level, and/or the binding of a binding species, as will be discussed in detail below.

A “binding species” refers to a species that binds to or otherwise associates with the M2 proton channel, affecting the degree to which protons can flow through the M2 channel. The binding is usually non-covalent. Typically, as previously discussed, the binding species may at least partially inhibit viral replication of the influenza virus. For instance, the binding species may be a molecule, a protein, a molecular complex, a small molecule (e.g., having a molecular weight of less than about 1,000 Da), or the like. The binding species may be, for instance, an adamantane-based binding species which includes, but is not limited to, amantadine and rimantadine. In certain instances, when a binding species is bound or otherwise associated with the channel, the channel is partially or completely closed, i.e., such that protons cannot flow through the M2 channel. In some cases, this binding stabilizes the closed conformation of the channel and inhibits viral replication. The binding of a binding species may also be weakened in certain cases because of mutations to the M2 tetramer, and the degree of binding, and the degree to which the M2 channel is closed, can be determined as discussed herein. In some cases, such as when the binding species is rimantadine, the binding species may bind to or otherwise associate with one or more residues located near the channel, for example, to Trp41.

The location in which the binding species binds to or otherwise associates with the M2 channel may affect the dynamics of the Trp41 residue. Therefore, it may be important in some embodiments to determine to what degree the binding species affects the M2 channel. The location of binding species binding may be determined, for instance, using nuclear magnetic resonance (NMR). Any suitable NMR technique may be used, and in some cases, multiple NMR techniques may be used. NMR techniques that may be employed include, but are not limited to, heteronuclear single quantum coherence (HSQC), nuclear Overhauser effect spectroscopy (NOESY), saturation transfer difference (STD) spectroscopy, and paramagnetic broadening enhancement (PBE). NMR techniques such as these will be known to those of ordinary skill in the art, and examples of suitable techniques can be found in the Examples, below.

As a specific non-limiting example, in some embodiments, when using ¹H—¹⁵N HSQC NMR, resonances that appear associated with a binding species may indicate which residues of the M2 channel are involved or associated with the binding of a binding species. For instance, in one embodiment, when a binding species such as rimantadine is present, the appearance of resonances for Leu43, Asp44, and/or Arg45 upon the binding of the binding species may indicate that these resonances are involved in the binding of the binding species to the M2 channel. In some cases, addition information about the association may be determined, for instance, using ¹³C-edited NOESY and/or ¹⁵N-edited NOESY NMR spectroscopy with ¹⁵N- and ²D-labeled protein, deuterated detergent, protonated rimantadine, etc. In certain cases, the presence of crosspeaks for Leu40 and/or Leu43 may indicate the location of the binding species. In addition, the appearance of backbone amide resonances upon the addition of the binding species for Ile43, Asp44, and/or Arg45 may also indicate further information about the location of the binding species, in some instances.

As mentioned, in one set of embodiments, the dynamics of the channel may be determined by NMR. For instance, timescale dynamics and/or the chemical exchange rate of protons or other species through the channel may be determined, e.g., as discussed herein. As an example, in certain embodiments, the dynamics and/or the chemical exchange rate of protons can be determined through observation of the Trp41 indole ring resonances. Relaxation-compensated Carr-Purcell-Meiboom-Gill (CPMG) experiments can be used, in some instances, to determine the timescale dynamics of the Trp41 ring between the closed and open states at different pH values. Those of ordinary skill in the art will be aware of relaxation-compensated CPMG experiments and how to conduct them. CPMG experiment may allow for the measurement of chemical or conformational exchange time constants, for instance, from approximately 10⁻⁵ to 10⁻¹ s.

A non-limiting example of such a CPMG technique follows. A basic pulse sequence of a CPMG experiment can be performed based on a ¹H—¹⁵N HSQC pulse sequence, in which the anti-phase 2H_{z}N_{y} magnetization after the first INEPT (insensitive nuclei enhanced by polarization transfer) is subject to a CPMG delay of duration T/2. It may be followed by another INEPT which converts the 2H_{z}N_{y} into the in-phase N_{X} magnetization, which is also subject to a CPMG delay of duration T/2. The N_{X} can be converted back to anti-phase for the gradient-selected HSQC readout. The timescale dynamics may be sampled by CPMG with a train of 180°¹⁵N pulses applied at varying frequencies, while the total duration of CPMG delays, T, can be kept constant. The decay of signal (R₂) vs the frequency of the 180°¹⁵N pulses (τ_{cp}) in the CPMG elements can be fitted to a hyperbolic tangent function for calculating the rate of two-state exchange (Kex) of the Trp41 residue. The Kex may be determined to determine the rate of the M2 channel opening. An example is discussed in the Examples, below.

The dynamics of the M2 channel may also be determined in the presence of a binding species and/or at different pH levels, such as those described below. In certain cases, the rate of fluctuation of the gate, e.g., between a relatively open state and a relatively closed state may increase or decrease, e.g., due to changes in pH and/or the addition of a binding species such as rimantadine.

In one aspect of the present invention, the degree that the M2 channel is open may be controlled by controlling the external environment. For instance, the M2 channel may be controlled by changes in pH, and/or by the presence of a binding species. For example, in one embodiment, the degree to which the M2 channel is open may be altered by a change in the pH level. The pH level may be altered using various methods which include, but are not limited to, the use of a buffer solution. Buffer solutions which may be employed include, but are not limited to, sodium phosphate. In some embodiments, the pH may be, for instance, about 6, about 7, or about 7.5. In some cases, the pH may be buffered between about 5.0 and about 8.3, or between about 7.5 and about 8.3. In yet other cases, the pH may be below about 7.5, or above about 8.3. As an example, in one embodiment, between the pH of about 7.5 and about 8.3, the closed conformation of the M2 channel may be stable over this region. As another example, at pH 7.5, the Trp41 indole rings may be positioned at van der Waals distance from each other and the passage or water, protons, or other ions through the channel can be partially or completely inhibited.

It is believed that alteration of pH may alter the formation or structure of the transmembrane helices of M2, and this can be determined, e.g., using NMR techniques such as those described herein. When the pH is lowered, the helix-helix packing within M2 may be at least partially destabilized. In some cases, this destabilization can be monitored through the broadening of resonances in an NMR spectrum. Broadening may be observed when the pH is lowered from about 7.5 to about 7.0, or when the pH is lowered from about 7.0 to about 6.0, etc.

In some instances, the degree to which the channel is open may be altered by a change in the pH in the presence of a binding species. For instance, the M2 channel may be studied at a first pH and a second pH, and in some cases, with and/or without the presence of the binding species. A change in pH may affect whether a binding species is able to bind to or otherwise associate with the M2 channel. At some pH levels, a binding species may not specifically bind to or associate with the M2 channel, e.g., such that the binding of the species to M2 is not specific to M2, although the binding species may be able to bind or associate with M2 at other pH levels. Thus, in one set of embodiments, the presence of a binding species at a certain pH level may affect the stability of the openness of the channel, and this can be determined, e.g., as described herein. For example, at pH 7.0, certain species such as rimantadine may be able to stabilize the channel in a relatively closed state. In some cases, such stabilization can be determined by the sharpening of resonances in the NMR spectra.

As mentioned, the addition of a binding species able to bind to or otherwise associate with the M2 channel may alter the dynamics of the M2 channel, e.g., proton conductance through the channel. Such dynamics can be determined, for instance, using NMR techniques such as those described herein. Accordingly, one aspect of the invention is generally directed to methods for evaluating binding species, for instance, to determine their suitability as a therapeutic agent for partially or completely inhibiting viral replication. For instance, by determining how the M2 channel is altered in the presence or absence of the binding species, e.g., by determining the dynamics of the tryptophan residue located in position 41 in the presence and/or absence of the binding species, the suitability of a binding species as a therapeutic agent may be determined.

In certain embodiments, M2 may be complexed or otherwise associated with a detergent, such as in a detergent micelle. Such a configuration may be useful for allowing M2 to retain a natural configuration, allowing its study using NMR techniques such as those described herein, as M2 is a transmembrane protein, as discussed. As is known to those of ordinary skill in the art, a detergent micelle is generally a thermodynamically stable colloidal aggregate of detergent monomers. Often, the nonpolar ends of the detergent molecules within a micelle are sequestered inward, avoiding exposure to water, and the polar ends of the detergent molecules are oriented outward in contact with the water. The M2 homotetramer may be complexed with a detergent for reasons which include, but are not limited to, solubility, stability, NMR signal quality and/or intensity of the M2 homotetramer. The detergent that may be employed includes, but is not limited to, phosphocholines such as dihexanoyl-phosphocholine (DHPC).

Water may be important for proton translocation for the M2 channel in some cases. Therefore, it may be important to determine information about water molecules that may be located in or near the M2 channel. Such information may include the number of water molecules within the M2 channel and/or the location of the water molecules within the M2 channel, and such information can be determined using techniques such as the NMR techniques described herein. For instance, the M2 channel may be complexed with a detergent micelle such as those described above, and the transmembrane region may be mostly protected from water; in such cases, it may be important to determine information about the water molecules associated with the channel, e.g., to determine the ability of the channel to conduct protons. The amount of water in the channel may vary, for instance, depending on factors such as pH, the binding species, or the like.

The following references are herein incorporated by reference: U.S. Provisional Patent Application Ser. No. 61/000,386, filed Oct. 25, 2007, entitled “Anti-Influenza Compositions,” by Chou, et al., and U.S. Provisional Patent Application Ser. No. 61/002,338, filed Nov. 8, 2007, entitled “Anti-Influenza Compositions,” by Chou, et al.

The following examples are included to demonstrate various embodiments of the invention. Those of ordinary skill in the art should, in light of the present disclosure, appreciate that many changes can be made in the specific embodiments which are disclosed and still obtain a like or similar result without departing from the spirit and scope of the invention. Accordingly, the following examples are intended to illustrate certain embodiments of the present invention, but do not exemplify the full scope of the invention.

### EXAMPLES

M2 is a 97-residue single-pass membrane protein with its N- and C-termini directed toward the outside and inside of the virion, respectively; it is believed to be a homotetramer in its native state. The channel region is formed by four transmembrane (TM) helices in which His37 is the pH sensor and Trp41 serves as the gate of the channel. The adamantane-based binding species, amantadine and rimantadine, which target the M2 channel, have been used as first-choice antiviral binding species against community outbreaks of influenza A viruses for many years, but resistance to the adamantanes has recently become widespread.

This example describes the structure of the tetrameric M2 channel in complex with rimantadine, determined by nuclear magnetic resonance (NMR) spectroscopy, in accordance with one embodiment of the invention. In the closed state at pH 7.5, it is believed that the channel gate is “locked” by inter-subunit hydrogen bonds between Trp41 and Asp44, which could be “unlocked” by lowering the pH. It is also believed that rimantadine binds at four equivalent sites near the gate on the lipid facing side of the channel-forming helices and stabilizes the closed conformation of the pore. Binding species-resistance mutations may change the properties of helix-helix packing, destabilize the closed state, and hence weaken binding species binding.

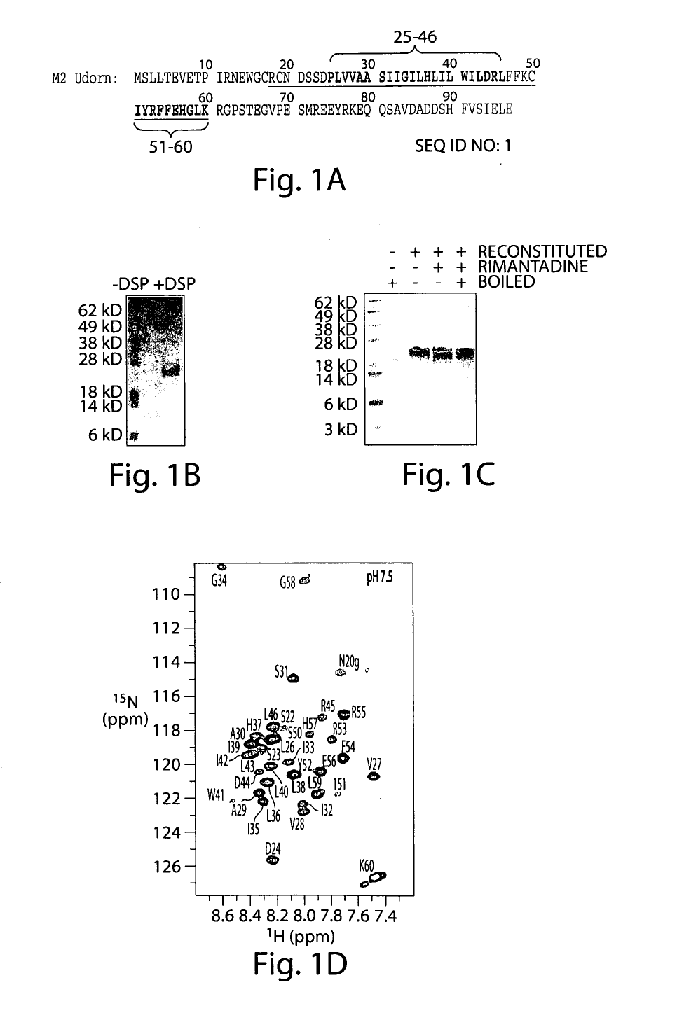

In these experiments, several constructs of varying length, ranging from the TM peptide to the full-length protein, were tested for tetramerization and NMR spectral quality in different detergent solutions. The region of residues 18-60 [M2(18-60)], which includes the TM domain as well as 15 residues of the C-terminal region, forms a stable tetramer in dihexanoyl-phosphocholine (DHPC) detergent micelles while yielding high-resolution NMR spectra. FIG. 1a shows the amino acid sequence of M2 (A/Udorn). Residues 18-60 are underlined. Cysteines 19 and 50 were mutated to serines. The TM and AP helical regions of the M2 are identified by the bolded residues of 25-46 and 51-60, respectively. At very low peptide concentrations (˜20 uM), chemical cross-linking using dithiobis(succinimidyl)propionate (DSP) resulted in a homogeneous tetramer. FIG. 1b shows the urea-PAGE of reconstituted M2(18-60) at very low concentration (20 uM monomer) with and without chemical cross-linking. Lanes from left to right are: 1) molecular weight (MW) markers, 2) NMR sample without DSP cross-linker, 3) the sample in lane 2 subject to 15 min of DSP cross-linking reaction.

At high protein concentrations used for the NMR experiments (0.75 mM monomer), M2(18-60) ran as a homogeneous tetramer in SDS-PAGE in the absence of cross-linkers, indicating a very stable assembly. FIG. 1c shows the SDS-PAGE of a typical NMR sample. Lanes from left to right are: 1) MW markers, 2) HPLC-purified M2(18-60) peptide dissolved directly into gel loading buffer solution followed by 5 min boiling, 3) NMR sample of M2(18-60) reconstituted using the protocol described below, 4) the sample used in lane 3 with rimantadine, and 5) the sample in 4 after 5 min of boiling, for demonstrating the stability of the tetrameric assembly.

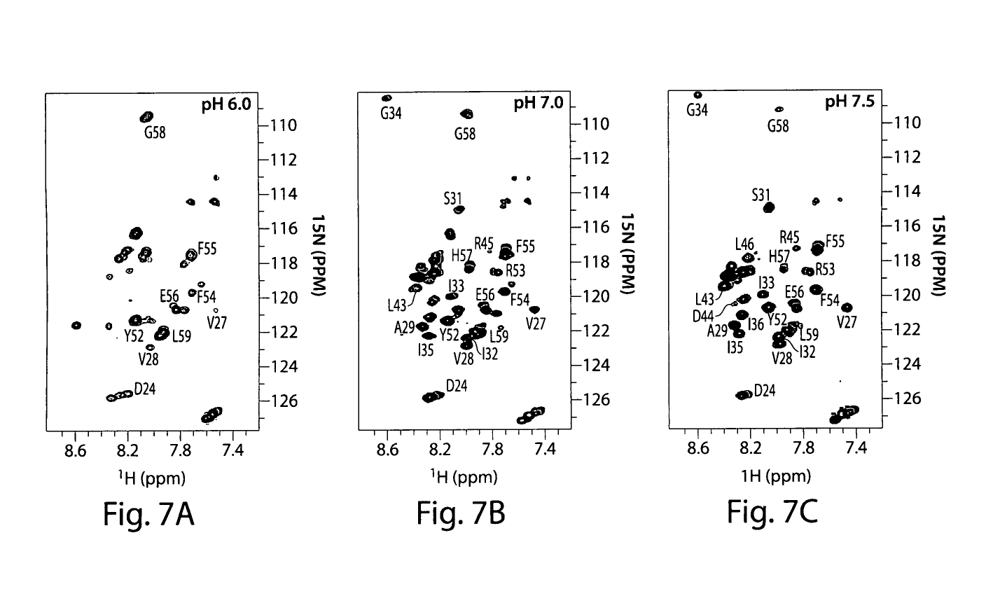

The addition of the binding species, rimantadine, improved the overall quality of NMR spectra and led to the appearance of three additional peaks corresponding to Leu43, Asp44, and Arg45 (FIG. 7). Conformational variability was evaluated by ¹H—¹⁵N heteronuclear single quantum coherence (HSQC) spectra in the pH range 5.0-8.3 in the presence of rimantadine. The TM region of the closed channel was significantly more stable than the open channel as HSQC resonances were much sharper and more homogeneous at high pH than at low pH (FIG. 7). The spectra did not change significantly between pH 7.4 and 8.3, indicating that the closed conformation was stable over this pH range. The spectra for structure determination was collected at pH 7.5 for the M2(18-60) tetramer in complex with rimantadine and DHPC micelles. The final sample conditions, which yielded a high-resolution ¹H—¹⁵N correlation spectrum, are described below. FIG. 1d shows a ¹H—¹⁵N transverse relaxation-optimized spectroscopy (TROSY) spectrum of uniform ¹⁵N-, 85% ²H-labeled M2(18-60) tetramer (0.75 mM monomer) in 300 mM DHPC and 40 mM rimantadine, recorded at 600 MHz ¹H frequency, pH 7.5, and 30° C. Each peak represented a backbone NH moiety with residue number labeled.

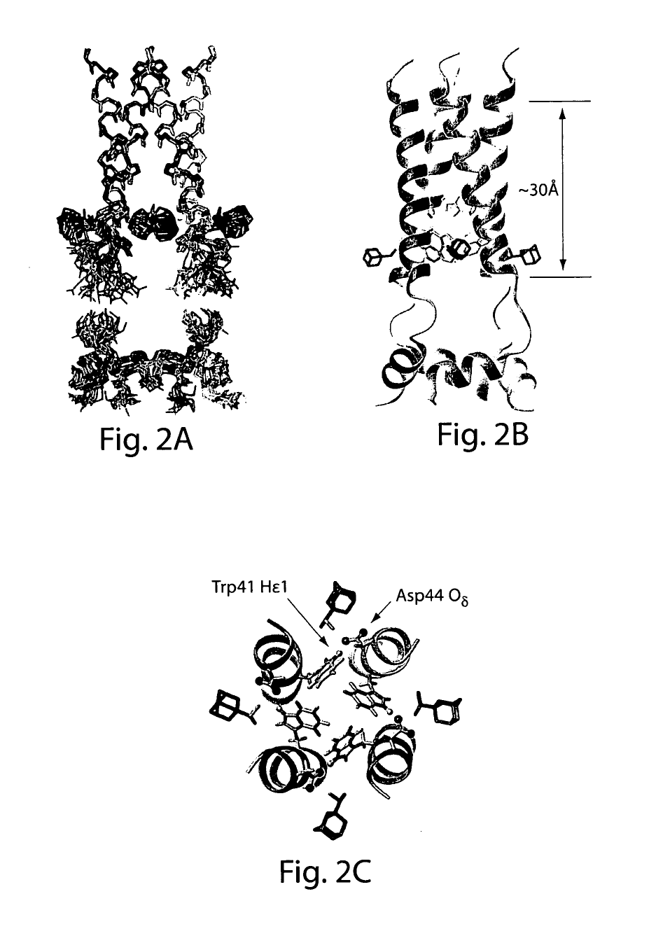

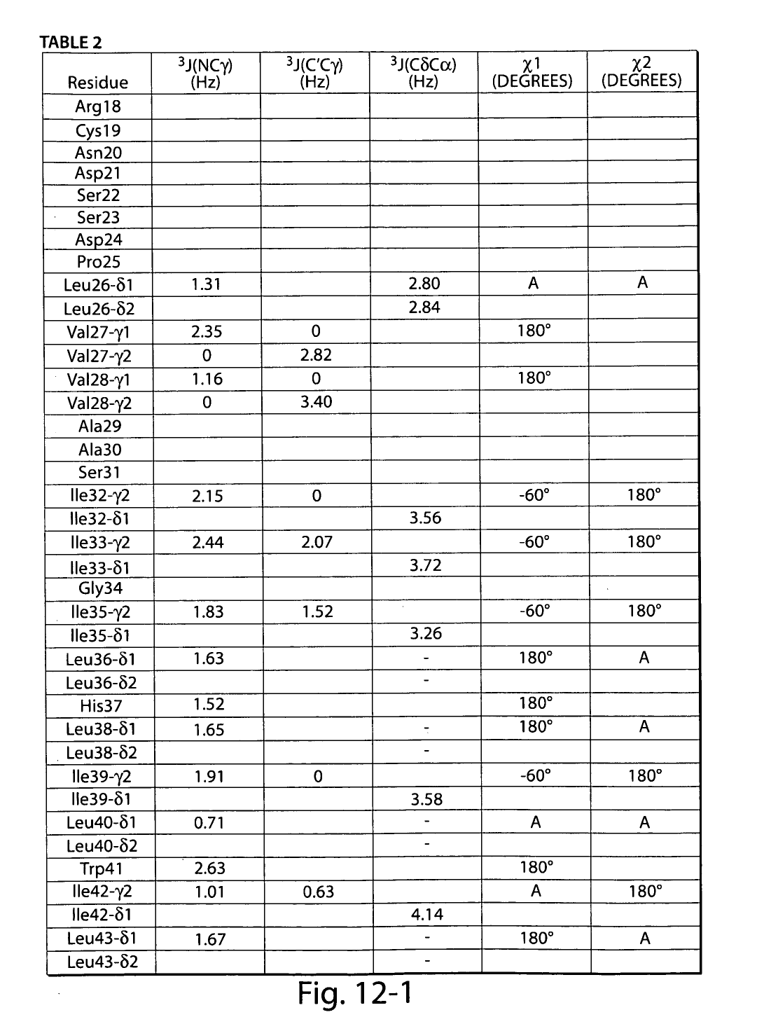

An extensive set of structural restraints including 230×4 intra- and 27×4 inter-molecular distance restraints derived from nuclear Overhauser enhancements (NOEs), 27×4 orientation restraints from residual dipolar couplings (RDCs), and 23×4 sidechain rotamers from 3-bond scalar couplings were used to generate an ensemble of 15 low energy structures with a backbone rmsd (root mean square deviation) of 0.30 Å for the channel region, and 0.89 Å for all structured regions. FIG. 2a shows the structure of the M2 channel. An ensemble of 15 low energy structures derived from NMR restraints (see below for details). Since residues 47-50 were unstructured, the TM helices (residues 25-46) and the AP helices (residues 51-59) are superimposed separately. The backbone rmsd for the TM and AP helices were 0.30 Å and 0.56 Å, respectively. FIG. 2b shows ribbon representation of a typical structure from the ensemble in FIG. 2a, showing the left-handed packing of the TM helices, right-handed packing of the AP helices, the sidechains of His37 and Trp41, as well as the binding species rimantadine. FIG. 2c shows a close-up view from the C-terminal side of the channel showing the Trp41 gate and how it was stabilized by the inter-monomer hydrogen bond between Trp41 Hε1 of one TM helix and Asp44 carboxyl of the adjacent TM helix. The refinement statistics and NMR-derived restraints are summarized in Table 1, given in FIG. 11.

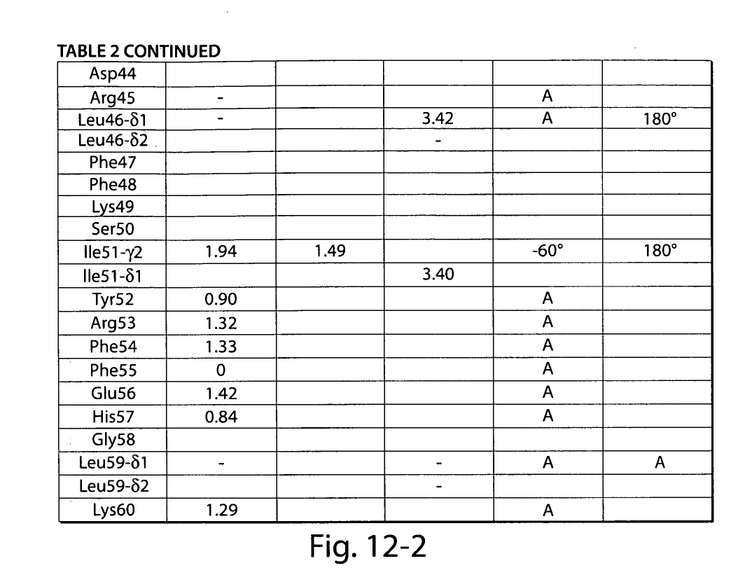

In the closed conformation at pH 7.5 and in the presence of rimantadine, M2 (18-60) is a homotetramer, in which each subunit has an unstructured N-terminus (residues 18-23), a channel-forming TM helix (residues 25-46), a short flexible loop (residues 47-50), and a C-terminal amphipathic (AP) helix (residues 51-59). The TM helices assemble into a four-helix bundle with a left-handed twist angle of ˜23° and a well-defined pore. A ring of methyl groups from Val27 constricts the N-terminal end of the pore to ˜3.1 Å (inner diameter). The sidechains of His37 and Trp41 were found to be inside the pore. According to the 3-bond ¹⁵N—¹³Cγ scalar coupling constant (³J_{NCγ}) of 1.5 Hz (see Table 2, given in FIG. 12), the His37 χ₁ rotamer was predominantly trans, but experienced significant rotameric averaging. The χ₁ of Trp41 is essentially locked in the trans position, as determined by ³J_{NCγ} of 2.6 Hz, while the χ₂ is also mostly fixed at around −120° by the sidechain Hε1-Nε1 RDC and NOEs. The Trp41 indole rings were at van der Waals (VDW) distance from each other, prohibiting water or ions from passing through. The indole Hε1 of one subunit was on average 3.5 Å from the Asp44 carboxyl carbon of the adjacent subunit. The two residues were in position to form an intermolecular hydrogen bond that stabilizes the Trp41 gate in the closed conformation. The C-terminal end of the channel extended into a loop (residues 47-50) that connected the TM domain to the C-terminal AP helix. RDCs and intra- and inter-monomer NOEs showed that the AP helices were oriented roughly perpendicular)(˜82° to the TM helices and were assembled head-to-tail with a right-handed packing mode to form the base of the channel. In this conformation, the only two lysines of the construct, Lys60 of one subunit and Lys49 of the adjacent subunit, were adjacent, showing that chemical cross-linking yielded pure circularly cross-linked tetramers rather than a distribution of oligomeric species. Residues 47-50 gave no NOE peaks and had no stable, hydrogen-bonded structure in the detergent micelles used in this work.

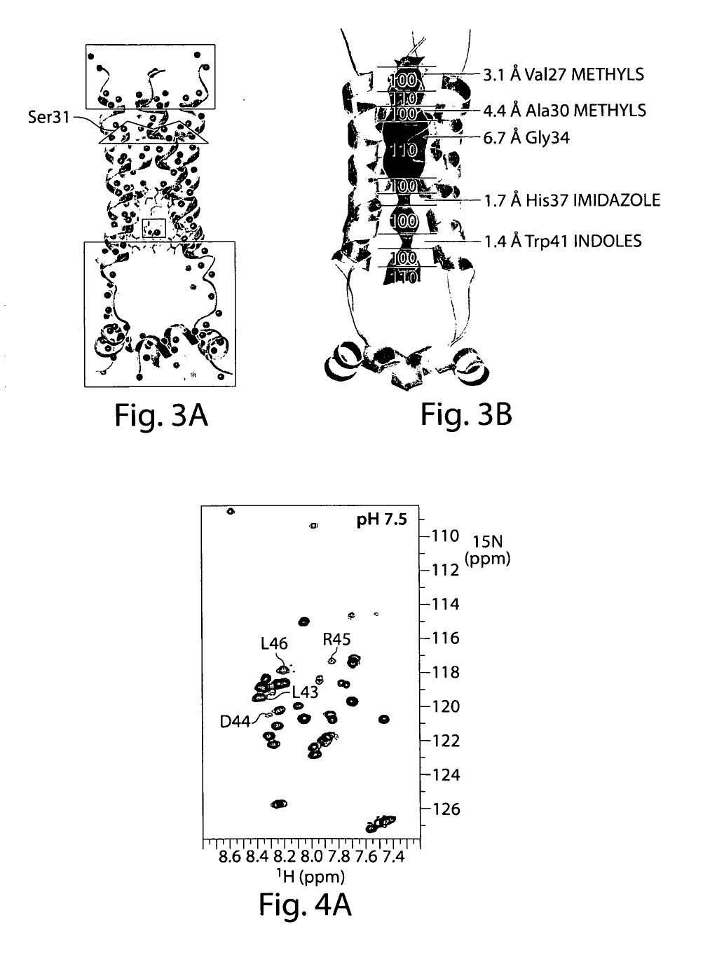

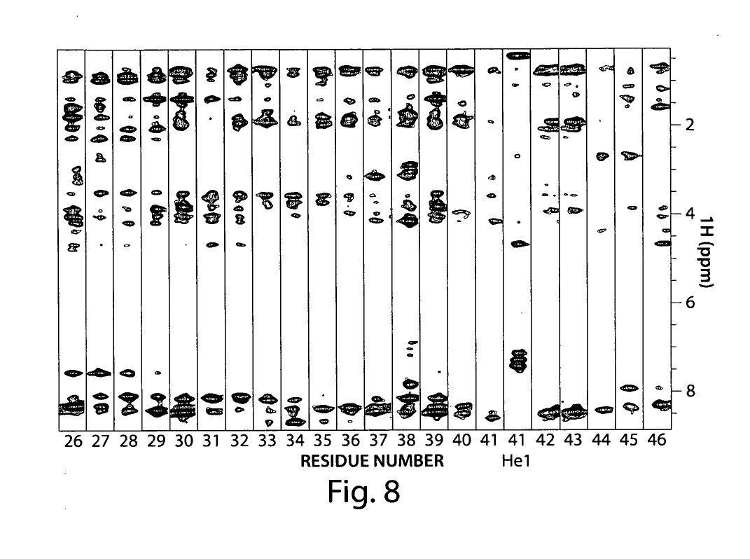

The TM region was largely protected from water by the DHPC micelle. Between the first turn, residues 26-28, and Leu46 at the C-terminus of the TM region, only the amides of Ser31 and Ile32 had NOE crosspeaks at the chemical shift of water in an ¹⁵N-edited NOESY spectrum with 110 ms mixing time (FIG. 8). The crosspeak at Ser31 corresponded to either the sidechain hydroxyl proton of Ser31 or the hydroxyl proton in exchange with water. By using a perdeuterated protein sample and increasing the NOE mixing time to 500 ms, the weak water crosspeaks to amide protons were observed as far as Ile33, suggesting that loosely bound water molecules may be present in the N-terminal half of the pore region in the closed channel. Water was detected at the C-terminus of the TM region, beginning at Arg45. The Hε1 of the Trp41 indole ring, which points toward the C-terminal side of the pore, also had a strong NOE to water, indicating that the base of the channel was accessible to bulk water. Water NOEs measured in the 110 ms ¹⁵N-separated NOESY experiments gave a picture of water distribution relative to the channel. FIG. 3a shows the water accessibility of the M2 channel and more specifically, the distribution of water NOEs relative to the structure. Amide protons that are located within the boxes have a NOE crosspeak to water. Those that do not are outside the boxes. FIG. 3b shows the pore surface calculated using the program HOLE. The region of the channel indicated by 100 is only wide enough to allow passing of a water molecule, where as the region of the channel indicated by 110 can accommodate two or more water molecules.

In the closed state, the Val27 ring at the N-terminus and the Trp41 gate at the C-terminus essentially blocked water from freely diffusing into the pore from both sides of the membrane. Inside the pore, water molecules concentrated at Ser31. The hydrated sidechain of Ser31 may serve to bridge the proton relay from the N-terminal end of the pore to the His37 pH sensor. A polar residue (Ser, Asn, or Lys) was present at position 31 in all sequenced variants of M2. The presence of a specific interaction with water at this position suggested that proton conduction may require water to be bound to this site.

Rimantadine-binding site was first determined using a ¹³C-edited NOESY with 150 ms mixing time. Distinct rimantadine NOEs were present for the methyl groups of Leu42 and Ile43. The sites were independently confirmed by recording a ¹⁵N-edited NOESY with 500 ms mixing time on a sample containing uniform ¹⁵N- and ²H-labeled protein, deuterated detergent, and protonated rimantadine. This spectrum contained strong and weak crosspeaks to the two magnetically identical adamantane protons at 1.58 ppm for the backbone amides of Leu40 and Leu43, respectively. The site assignment is consistent with the appearance of Ile43, Asp44, and Arg45 backbone amide resonances upon addition of rimantadine.

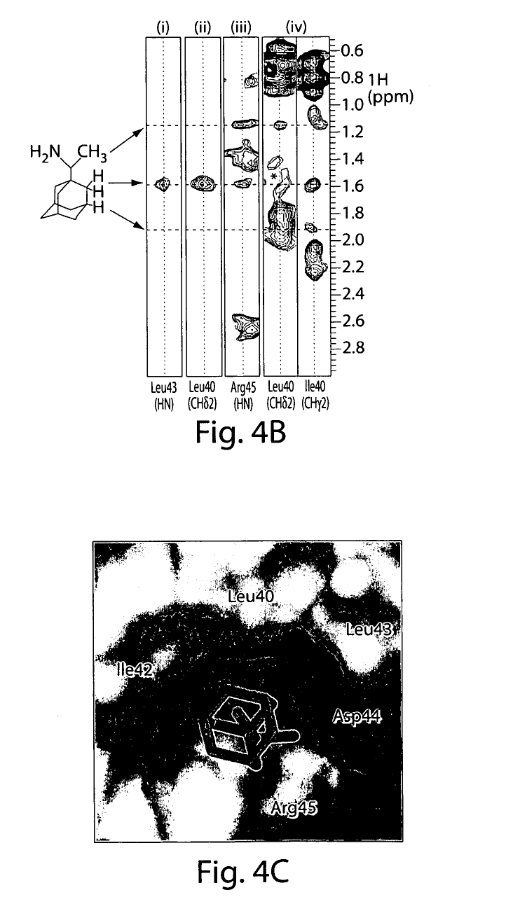

FIG. 4 shows the interaction between rimantadine and the M2 channel. FIG. 4a specifically shows the overlay of the ¹H-⁻¹⁵N TROSY spectra of reconstituted M2(18-60) tetramer at pH 7.0 in the absence (black) and presence (grey) of rimantadine, recorded at 500 MHz ¹H frequency and 30° C. Labeled resonances are those which became significantly more intense upon rimantadine addition. FIG. 4b shows the selected strips showing intermolecular NOEs between the protein and rimantadine. Experiments are (i) ¹⁵N-separated NOESY (500 ms NOE mixing) on ¹⁵N-, ²H-labeled M2(18-60), (ii) ¹³C-filtered, ¹³C-separated NOESY (200 ms mixing) on ¹⁵N-, ¹³C-labeled M2(18-60), (iii) ¹⁵N-separated NOESY (110 ms mixing), and (iv) ¹³C-separated NOESY (150 ms mixing). FIG. 4c shows the surface representation of the rimantadine binding pocket, showing the Asp44, the indole amine of Trp41, and Arg45 that form the polar patch, as well as the hydrophobic wall composed of Leu40, Ile42, and Leu43.

The protein-binding species NOEs collected from four different NOESY spectra placed the binding site between adjacent helices at the C-terminal end of the TM domain near the Trp41 gate, on the membrane side of the channel. The amine head-group of rimantadine was in contact with the polar sidechains of Asp44, Arg45, and the indole amine of Trp41. The sidechains of Ile42 formed one helix, and Leu40 and Leu43 from another helix formed the hydrophobic walls of the binding pocket that interact with the adamantane group of rimantadine.

Simple 2D NMR spectra of M2(18-60) with and without rimantadine at different pH values suggest that binding species binding stabilizes the closed conformation. Upon addition of rimantadine at pH 7.0, the linewidth of NMR resonances in the ¹H—¹⁵N correlation spectrum become significantly sharper and more homogeneous, and the resonances of residues 43-45 at the C-terminus of the TM helix, absent in the binding species-free sample, became observable. At pH 7.5, addition of rimantadine perturbed a specific set of residues but did not substantially improve the spectra, as the channel was predominantly closed above pH 7.4. At pH 6.0, where a significant fraction of the channel may be expected to be open, adding the binding species had a minor effect on the spectrum, suggesting that rimantadine has much lower affinity, if any, for the open state.

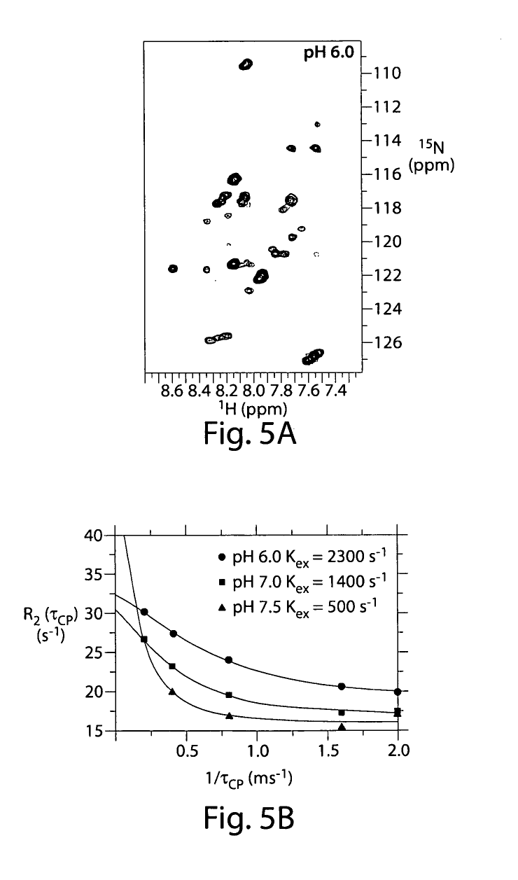

Lowering the pH from 7.5 to 7.0 and from 7.0 to 6.0 both broadened most of the resonances in the ¹H—¹⁵N correlation spectrum corresponding to the channel-forming TM helix. FIG. 5 shows the low-pH induced destabilization of the channel and opening of the Trp41 gate. FIG. 5a specifically shows the overlay of the ¹H—¹⁵N TROSY spectra of reconstituted M2(18-60) tetramer at pH 6.0 in the absence (black) and presence (grey) of rimantadine, recorded at 500 MHz ¹H frequency and 30° C. In both spectra, the resonances corresponding to the TM helix either have disappeared or were perturbed beyond recognition. FIG. 5b shows the ¹⁵N R₂ (pure R₂+Rₑₓ) of the Trp41 Nε1 as a function of the frequency of refocusing (1/τ_{cp}) of chemical shift evolution obtained at pH 7.5, 7.0, and 6.0, showing faster timescale motion of the Trp41 gate as the channel is activated. FIG. 5c shows the comparison between R₂(τ_{cp}) at pH 7.0 in the absence (triangles) and presence (squares) of rimantadine, demonstrating that the binding species slowed down the gate flipping at this pH. The resonance broadening observed could not be attributed to protein aggregation, as the ¹⁵N R₁ and R₂ relaxation rates and self-diffusion coefficients were essentially unchanged between pH 7.5 and 7.0.

Thus, activation of the channel may be coupled to increased conformational exchange in the TM domain. In contrast, the resonances of the AP helices were essentially unaffected by lowering the pH, indicating that the C-terminal base of the tetramer remained intact. Electrostatic repulsion between the protonated His37 imidzole rings may be the initial step in low pH activation. Disruption of inter-helical contacts could increase the pore size and admit water to conduct protons across the membrane. When the TM helices were repelled in the open state, the tetrameric complex was kept intact both by the C-terminal base and by disulfide bonds at the N-terminus. Truncation of the AP helix resulted in channels that rapidly lose channel activity.

In addition to destabilizing helix-helix packing in the TM domain, channel activation may also correlate with increased dynamics of the Trp41 gate. The indole amide resonance of Trp41 remained intense as the pH was lowered from 7.5 to 6.0, and it served as a useful NMR probe for monitoring channel opening. The ms timescale dynamics of the Trp41 indole ring between the closed and open states was compared by carrying out relaxation-compensated Carr-Purcell-Meiboom-Gill (CPMG) experiments at pH 7.5, 7.0, and 6.0. As shown in FIG. 5b, a two-site exchange model fit the dependence of ¹⁵N relaxation due to chemical shift exchange on the frequency of refocusing (1/τ_{cp}) of chemical shift evolution. This result implied that the gate was able to switch between two configurations at any given pH. As the pH was lowered from 7.5 to 6.0, the rate of fluctuation increased by more than four-fold (FIG. 5b), indicating that the gate was “unlocked” upon channel activation. Adding rimantadine to the channel at an intermediate pH of 7.0 slowed the timescale of the gate motion to that of a binding species-free gate at pH 7.5 (FIG. 5c). These results confirmed that the reconstituted channels in the NMR sample are pH-gated, and were consistent with the location of the rimantadine site proximal to Trp41. The binding species may stabilize the gate directly, or through Asp44.

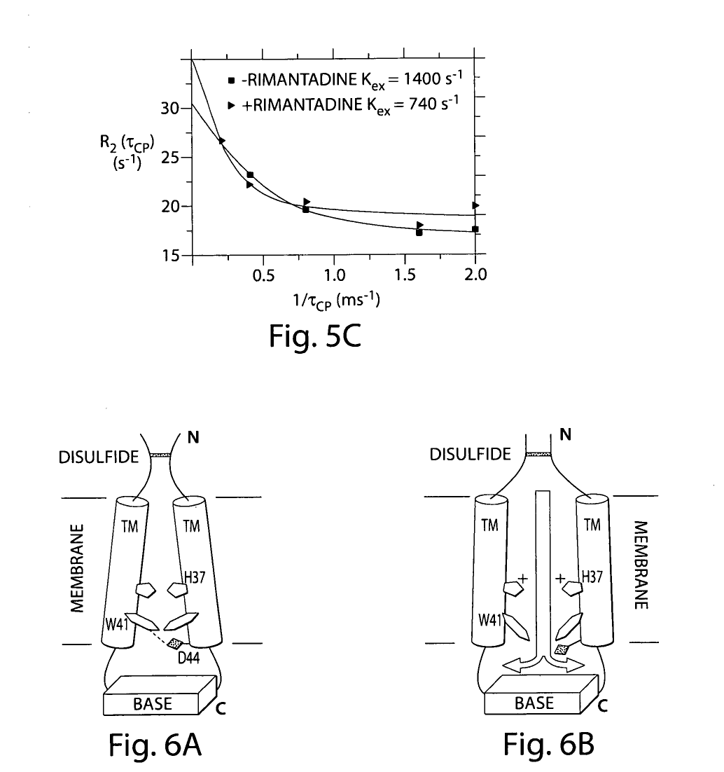

FIG. 6 shows a schematic illustration of M2 channel activation. At high pH, the TM helices were packed tightly and the tryptophan gate was locked through intermolecular interactions with Asp44. At low pH, protonation of the His37 imidazoles destabilized the TM helix packing, allowing hydration of the channel pore, and proton conductance. The C-terminal base of the tetramer and N-terminal disulfide bonds kept the channel from completely disassembling. For clarity, only two of the four monomers are shown.

### Sample Preparation

To prepare the sample for these experiments, M2(18-60) was expressed into inclusion bodies as a fusion to (His)₉-trpLE. The M2(18-60) peptide was released from the fusion protein by CNBr digestion in 70% formic acid (2 hr, 0.2 g/ml). The digest was dialyzed to water, lyophilized, and loaded onto a C4 column (Grace-Vydac) in 2:1:2 hexafluoroisopropanol:formic acid:water and separated on a gradient of 3:2 isopropanol:acetonitrile. The lyophilized peptide was refolded at 250 uM by dissolving it in 6 M guanidine and 150 mM DHPC and dialyzing against the final NMR buffer containing 40 mM sodium phosphate and 30 mM glutamate. The sample was concentrated to a final M2(18-60) concentration of 0.75 mM (monomer). Rimantadine was added after concentrating. The concentration of DHPC was determined from ¹H NMR spectroscopy to be around 300 mM. Given the DHPC has an aggregation number of 27 and the strong partition coefficient of rimantadine in phospholipid, there were approximately four rimantadine molecules per complex of M2 channel and DHPC micelle.

### NMR Spectroscopy

Data processing and spectra analyses for this experiment was completed in NMRPipe and CARA. The program TALOS was used to predict backbone dihedral angles from characteristic chemical shifts. Fitting of residual dipolar couplings (RDCs) to structures was done by singular value decomposition (SVD), using the program PALES. Analysis of the relaxation-compensated CPMG experiment was done using the program CPMGfit from Art Palmer

Complete sequence specific assignment of backbone ¹H^{N}, ¹⁵N, ¹³C^{α}, and ¹³C^{β} chemical shift were accomplished by performing a suite of standard triple resonance experiments, including the TROSY version of HNCA and HNCACB on a ¹⁵N-, ¹³C, and 85% ²H-labeled protein sample at 600 MHz ¹H frequency. Having the residue-specific chemical shift of ¹H^{N} and ¹⁵N, a 3D ¹⁵N-edited NOESY, recorded with 110 ms mixing time on a sample containing uniform ¹⁵N-, ¹³C-labeled protein, rimantadine, and deuterated DHPC (1,2-Dihexanoyl(D22)-sn-Glycero-3-Phosphocholine-1,1,2,2-D4-N,N,N-trimethyl-D9) (Avanti Polar Lipids, Inc.), was used to correlate the backbone amide and sidechain aliphatic and aromatic ¹H resonances. Since the amide resonances were well resolved and all structured regions of M2(18-60) are α-helical, as indicated by ¹³C^{α} and ^{—}C^{β} chemical shifts (TALOS) and the characteristic local NOE patterns of a helix, assignment of intra-residue and sequential NOEs in the ¹⁵N-separated NOESY spectrum was straightforward.

FIG. 7 shows the overlay of the ¹H—¹⁵N TROSY spectra of reconstituted M2(18-60) tetramer in the absence (black) and presence (grey) of rimantadine at pH 6.0, 7.0, and 7.5, recorded at 500 MHz ¹H frequency and 30° C. FIG. 8 is an image of the 1H-¹⁵N strips corresponding to residues 26-46 from the 3D ¹⁵N-edited NOESY with water-gate readout pulse scheme, recorded at ¹H frequency of 600 MHz on a sample containing 0.75 mM (monomer) M2(18-60), 40 mM rimantadine, 300 mM D35-DHPC, 40 mM sodium phosphate (pH 7.5) and 30 mM glutamate. The spectrum was acquired with 110 ms NOE mixing time, 36 ms of ¹⁵N evolution and 19 ms ¹H evolution in the indirect dimension. Using the same approach, the assigned chemical shifts of aliphatic and amide protons were then used to assign the methyl ¹H and ¹³C resonances, which were also mostly resolved in a 2D ¹H—¹³C HSQC spectrum recorded with 28 ms constant-time (CT) ¹³C evolution.

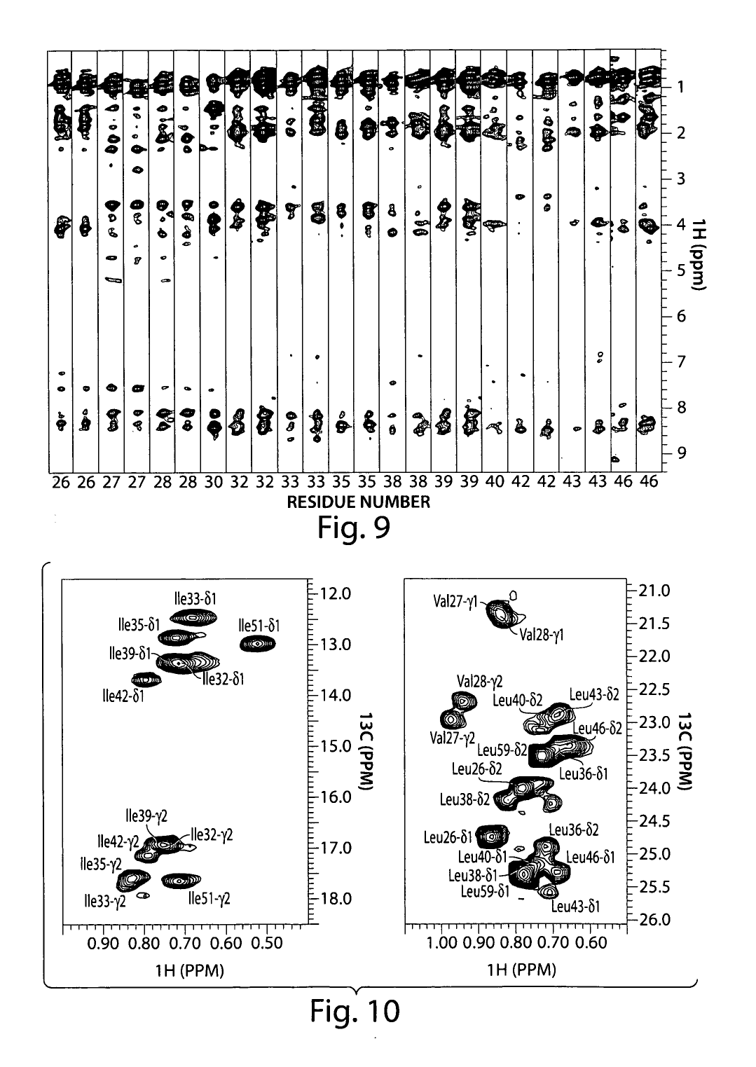

FIG. 9 shows the ¹H—¹³C strips corresponding to the methyl groups of residues 26-46 from the 3D ¹⁵C-edited NOESY with gradient coherence selection, recorded at ¹H frequency of 600 MHz on a sample containing 0.75 mM (monomer) M2(18-60), 40 mM rimantadine, 300 mM D35-DHPC, 40 mM sodium phosphate (pH 7.5) and 30 mM glutamate. The spectrum was acquired with 150 ms NOE mixing time, 28 ms constant-time ¹³C evolution, and 26 ms ¹H evolution in the indirect dimension. Specific stereo assignment of the gamma ¹³C of valine and delta ¹³C of leucine were obtained from a 10% ¹³C-labeled protein sample by recording a ¹H—¹³C HSQC with 28 ms CT ¹³C evolution as previously described.

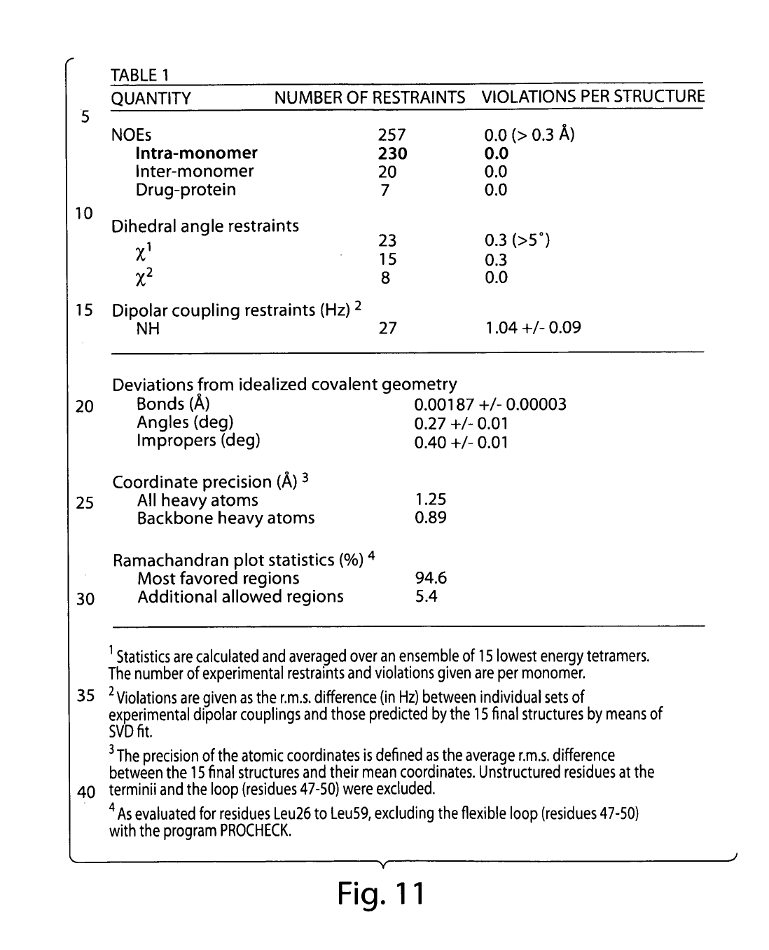

FIG. 10 shows the methyl regions of a high-resolution ¹H—¹³C HSQC spectrum recorded with 28 ms of constant-time ¹³C evolution using a sample containing 0.75 mM (monomer) M2(18-60), 300 mM D35-DHPC, 40 mM rimantadine, and 30 mM glutamate in a 40 mM sodium phosphate buffer with a pH of 7.5. The labels on the spectrum are the complete assignments of methyl resonances. This was accomplished using a 3D ¹³C-edited NOESY, recorded with 150 ms mixing time and 28 ms of CT ¹³C evolution on the same sample with deuterated detergent.

For determining whether the sidechain χ₁ rotamers were trans for amino acids other than Thr, Val, and Ile, ³J_{NCγ} coupling constants were measured using 2D spin-echo difference methods based on the ¹H—¹⁵N constant-time TROSY experiment performed on ¹⁵N-, ¹³C-, and 85% ²H-labeled protein at ¹H frequency of 750 MHz. For ³J_{C′Cγ} and ³J_{NCγ} of Thr, Val, and Ile, and ³J_{CαCδ} of Leu and Ile, 2D spin-echo difference methods based on ¹H-¹³C constant-time HSQC experiments were employed. These spectra were recorded at ¹H frequency of 600 MHz. Three-bond J couplings used for assigning sidechain χ₁ and χ₂ rotamers are given in Table 2 shown in FIG. 12. The capital letter ‘A’ indicates rotameric averaging for which no dihedral restraints were used during structure calculation. Rotamer information was extracted from the couplings according to analyses previously described

Weak alignment of the DHPC-reconstituted M2 relative to the magnetic field was accomplished using a modified approach of the strain-induced alignment in a gel (SAG) method. The protein/detergent solution was soaked into a cylindrically shaped polyacrylamide gel (4.5% acrylamide concentration and acrylamide/bisacrylamide molar ratio of 80), initially of 6 mm diameter and 9 mm length, which was subsequently radially compressed to fit within the 4.2 mm inner diameter of a NMR tube with open ends. The RDCs were obtained from subtracting J of the unaligned sample from J+D of aligned sample. The sign of dipolar couplings follows the convention that |¹J_{NH}+¹D_{NH}|<90 Hz when ¹D_{NH} is positive. The ¹H—¹⁵N RDCs were obtained from ¹J_{NH}/2 and (¹J_{NH}+¹D_{NH})/2, which were measured at 600 MHz (¹H frequency) by interleaving a regular gradient-enhanced HSQC and a gradient-selected TROSY, both acquired with 80 ms of ¹⁵N evolution. On the basis of the length of the time domain data and the signal to noise ratio, the accuracy of the measured RDCs was expected to be at ±0.5 Hz (¹D_{NH}).

### Structure Determination

Structure calculation was completed using the program XPLOR-NIH. The secondary structure of the monomer was first calculated from random coil using intramonomer NOEs, backbone dihedral restraints derived from chemical shifts (TALOS) and sidechain χ₁ and χ₂ restraints shown in Table 2 given in FIG. 12. This was done using the following high-temperature simulated annealing (SA) protocol. The intramonomer NOE restraints were enforced by flat-well harmonic potentials, with the force constant fixed at 50 kcal mol⁻¹ Å⁻². For sidechain χ₁ and χ₂ angles that were not assigned as rotamer averaging in Table 2 given in FIG. 12, flat-well)(±30°) harmonic potentials were applied with force constant fixed at 30 kcal mol⁻¹ rad⁻². Other force constants, commonly used in NMR structure calculation, were: k(vdw)=0.02 →4.0 kcal mol⁻¹ Å⁻², k(impr)=0.1→1.0 kcal mol⁻¹ degree⁻², and k(bond angle)=0.4→1.0 kcal mol⁻¹ degree⁻². During the annealing run, the bath was cooled from 1000 to 200 K with a temperature step of 20 K, and 6.7 ps of Verlet dynamics at each temperature step, using a time step of 3 fs. A total of 20 monomer structures were calculated using this protocol. The lowest energy structure was chosen for subsequent tetramer assembly.

To obtain an initial set of tetramer structures, four copies of the lowest-energy subunit structure calculated above were used. A high-temperature SA protocol similar to that of above was performed in the presence of intermonomer NOEs and all other intramonomer restraints except RDCs. For each experimental intermonomer NOE between two adjacent subunits, four identical distance restraints were assigned respectively to all pairs of neighboring subunits to satisfy the condition of C4 rotational symmetry. These restraints were enforced by flat-well (±0.6 Å) harmonic potentials, with the force constant ramped from 25 to 100 kcal mol⁻¹ Å⁻². During the annealing run, the bath was cooled from 1000 to 200 K with a temperature step of 20 K, and 6.7 ps of Verlet dynamics at each temperature step, using a time step of 3 fs. A total of 100 tetramer structures were generated for independent validation by RDCs.

The 100 structures calculated above were independently validated by ¹H—¹⁵N RDCs. Fitting of RDCs to structures was done by singular value decomposition (SVD), using the program PALES. The goodness of fit was assessed by both Pearson correlation coefficient (r) and the quality factor (Q). Among the 100 structural models, 15 structures of which the individual subunits have on average the best agreement with RDCs (r˜0.91 and Q_{free}˜0.25) have been selected for a second round, low-temperature refinement against RDCs in the presence of all other NOE and dihedral restraints. For RDC refinement in the XPLOR-NIH program, approximate initial values of the magnitude (Dₐ) and rhombicity (Rₕ) of the alignment tensor were required. The average Dₐ (14.0 Hz) and Rₕ (0.20) obtained from the best SVD fits. In theory, for a tetramer with a C4 rotational symmetry, the alignment tensor is axially symmetric, or Rₕ=0. Deviation from an axially symmetric tensor was also observed in the phospholamban homo-pentamer in some cases. Since Rₕ≠0, the RDCs assigned equivalently to the four subunits could not be refined against a single alignment tensor. Instead, in this example, the RDCs of the four subunits were subject to four separate alignment tensors during XPLOR refinement.

During the refinement, the bath was cooled from 200 to 20 K with a temperature step of 10 K, and 6.7 ps of Verlet dynamics at each temperature step, using a time step of 3 fs. The force constants for NOE and experimental dihedral restraints were fixed at 100 kcal mol⁻¹ Å⁻² and 40 kcal mol⁻¹ rad⁻², respectively. In addition to the experimental χ₁ and χ₂ restraints, a weak database-derived ‘Rama’ potential function in XPLOR was ramped from 0.02 to 0.2 (dimensionless force constant) for the general treatment of sidechain rotamers. RDC restraint force constant was ramped from 0.01 to 0.125 kcal mol⁻¹ Hz⁻² (normalized for the ¹D_{NH} couplings). All other force constants were the same as before. For each of the 15 structures validated by RDCs, an ensemble of 10 RDC-refined structures were generated and the structure with the lowest total energy was chosen to represent the ensemble. In the end, 15 structures were obtained, each having the lowest energy in its corresponding ensemble, for describing the structural diversity of the NMR structure.

### Measurement of Chemical Exchange in the Trp41 Indole Ring

In the same embodiment, the timescale of chemical shift exchange of the Trp41 sidechain was measured using a relaxation-compensated CPMG experiment in ID mode at ¹H frequency of 600 MHz. The dependence of ¹⁵N relaxation due to chemical exchange on the frequency of refocusing (1/τ_{cp}) of chemical shift evolution was fitted to a two-site exchange model given by Rₑₓ∝1−(2τ_{ex/τ}_{cp})tanh (τ_{cp}/2τₑₓ), where Rₑₓ is the contribution to transverse relaxation due to chemical shift exchange, and τₑₓ is the correlation time of the process that is generating the chemical shift exchange.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
determining the dynamics of the tryptophan residue located in position 41 of the M2 proton channel of the influenza A virus via nuclear magnetic resonance.

2. The method of claim 1, comprising determining the probability of channel opening of the M2 proton channel by determining the dynamics of the tryptophan residue located in position 41.

3. The method of claim 1, comprising determining the chemical exchange rate of the indole amide of the tryptophan residue.

4. The method of claim 1, wherein the chemical exchange rate are determined using a relaxation-compensated Carr-Purcell-Meiboom-Gill experiment.

5. The method of claim 1, wherein the chemical exchange rate are determined by determining the R₂ relaxation time constant of ¹⁵N as a function of the frequency of refocusing of the chemical shift evolution.

6. The method of claim 1, comprising determining the chemical exchange rate of the tryptophan residue at a first pH and at a second pH substantially different from the first pH.

7. The method of claim 6, comprising determining the probability of channel opening with respect to pH.

8. A method of evaluating a candidate binding species, comprising:
exposing the M2 proton channel of the influenza A virus to a candidate binding species; and
determining whether the candidate binding species alters the dynamics of the tryptophan residue located in position 41 of the M2 proton channel of the influenza A virus.

9. The method of claim 8, wherein the dynamics is determined by determining the chemical exchange rate of the indole amide of the tryptophan residue via nuclear magnetic resonance.

10. The method of claim 8, wherein the relaxation kinetics are determined using a relaxation-compensated Carr-Purcell-Meiboom-Gill experiment.
